(19) United States
(12) Patent Application Publication
(10) Pub. No.: US 2025/0318824 A1
(43) Pub. Date: Oct. 16, 2025

(54) SUTURE LOCKING CINCH SYSTEM

(71) Applicant: EndoFix Medical Technologies, Inc., Plainville, MA (US)

(72) Inventors: Rickey Hart, Wrentham, MA (US); William Blenkhorn, Medway, MA (US)

(21) Appl. No.: 19/177,168

(22) Filed: Apr. 11, 2025

Related U.S. Application Data
(60) Provisional application No. 63/633,499, filed on Apr. 12, 2024.

(51) Int. Cl. A61B 17/04; A61B 17/00

(52) U.S. Cl. CPC A61B 17/0401; A61B 17/00234; A61B 2017/00296; A61B 2017/00367; A61B 2017/00477; A61B 2017/0409; A61B 2017/0446

(57) ABSTRACT

A suture locking cinch system can be used for surgically cinching and terminating a suture anywhere in the body. This suture locking cinch system includes a instrument that approximates a suture lock to secure the suture. The suture lock is necessary when a suture is passed through two or more pieces of tissue, and requires a locking device in place of tying knots. Specifically, for endoscopic surgery, a suture (e.g., a monofilament type suture) emanating from inside the body is threaded through a suture lock at a distal tip of the instrument, and the instrument is placed down an endoscope. The suture lock is forwarded to the locking site and with the suture being pulled taut, a handle assembly is depressed which deploys the suture lock (e.g., locking mechanism). The locking mechanism is severed from the instrument shaft and the suture is subsequently cut all in one motion.

### PRIORITY

This patent application claims priority from provisional U.S. patent application No. 63/633,499, filed Apr. 12, 2024, entitled, “SUTURE LOCKING CINCH SYSTEM,” and naming Rickey Hart and William Blenkhorn as inventors, the disclosure of which is incorporated herein, in its entirety, by reference.

### FIELD

Illustrative embodiments of the invention generally relate to surgical suture fixation and, more particularly, various embodiments of the invention relate to devices, systems, and methods of deploying the locking mechanisms to lock the suture after a fixation procedure.

### BACKGROUND

Surgical Fixation devices have primarily been manufactured from stainless, titanium, non-absorbable plastic, absorbable plastic or some hybrid of plastic and ceramic. However, the use of flexible fixation devices is far less common.

There are medical treatment settings where there is fixation of soft tissues, such as closing gastroenterological defects. In settings where sutures are used, devices that can lock sutures in place could be used for any soft tissue to tissue approximation.

### SUMMARY OF VARIOUS EMBODIMENTS

In accordance with one embodiment of the invention, a suture locking cinch system includes a handle assembly. The handle assembly includes a handle front and a handle back. The system also includes an outer shaft. The outer shaft is configured to provide a flexible hollow tube. A proximal end of the outer shaft is removably connected to the handle front. The system also includes a pull rod. The pull rod is removably attached to the handle back and configured to be run through a channel in the handle front and through the outer shaft. The system also includes a suture lock tip assembly. The suture lock tip assembly is configured to be frangibly removable to a distal end of the outer shaft. The system may also include a threader assembly. The threader assembly may be configured to be removably coupled to the outer shaft.

The threader assembly may include a suture threader line. The suture threader line may have a closed loop on a distal end the suture threader line. The threader assembly may include a threader tab. A proximal end of the suture threader line may be configured to be removably attached to a distal end of the threader tab.

The suture lock tip assembly may include an inner cutter assembly, an outer cutter assembly, and a suture lock assembly. The outer cutter assembly may be fixed into a distal end the outer shaft. The inner cutter assembly may be mounted in the outer cutter assembly. The suture lock assembly may be mounted in the inner cutter assembly. The suture lock assembly may include a suture lock outer and an inner post.

The inner post may be positioned in the suture lock outer. The pull rod may be coupled to a proximal end of the inner post. The inner post may include ribs. The ribs may be configured to provide an interference fit against the suture lock outer. The inner post may include a breakaway portion, a shearing under cut, and a shear point. The inner post may be configured to breakaway at the shear point when a shear force is applied by the pull rod. The shear point may be configured to breakaway when the shear force exceeds about 6 pounds.

The inner cutter assembly may include an inner cutter. The inner cutter may include at least one tab on an inner surface of inner cutter, and the at least one tab may be configured to engage the breakaway portion of the inner post.

The handle front and handle back may be molded with a boss feature that snaps together. The boss feature may be configured to movably join the handle front to the handle back and to provide a pivot hinge about which the handle front and handle back rotate.

The handle assembly further includes a dowel pin. The dowel pin may be configured to movably join the handle front to the handle back and to provide a pivot hinge about which the handle front and handle back rotate.

In accordance with another embodiment of the invention, an inner post with a shear point for placement and release of a suture lock includes a breakaway portion. The breakaway portion is configured to be engaged by a hooking portion.

The inner post includes a shearing undercut. The shearing undercut is configured to provide an undercut for defining the breakaway portion; and

The inner post also includes a shear point located at a junction on the inner post between the breakaway portion and the shearing undercut. The shear point is configured to allow the breakaway portion to be separated from the shearing undercut.

The inner post may further include an opening in the inner post. The opening may pass through the inner post at a right angle to the longitudinal direction of the inner post. The opening may be configured to allow the passage of a suture through the opening from one side of the inner post to the opposite side of the inner post.

The inner post may further include a suture lock outer. The suture lock outer may be configured to receive the inner post. The inner post may be configured to be slidably inserted into the suture lock outer.

The inner post may further include one or more ribs encircling the inner post. The one or more ribs may encircle the inner post radially at a right angle to the longitudinal direction of the inner post. The ribs may be configured to engage an inside surface of the suture lock outer.

In accordance with another embodiment of the invention, a method of surgically cinching and terminating a suture includes pulling the suture through an open loop of a suture threader line. The suture threader line is removably attached to a distal end of a threader tab. The method also includes pulling the suture threader line through a suture lock. The method also includes inserting the suture lock through an endoscope. The suture lock being positioned at the distal end of the outer shaft.

The method also includes approximating the suture lock to the tissue, and applying tension to the suture. The tension on the suture is applied by pulling the suture.

The method also includes removing a safety from a handle assembly, and squeezing the handle assembly to secure an inner post of the suture lock. The method also includes separating, frangibly, the suture lock while cutting the suture, and withdrawing the outer shaft along with the cut suture.

The pulling the suture through an open loop of a suture threader line may be performed outside of an endoscope.

Squeezing the handle assembly may pull the inner post in a proximal direction. A shear force may be created at a shear point in the inner post that separates a breakaway portion of the inner post from a shearing under cut of the inner post at the shear point. Squeezing the handle assembly may pull the inner post in a proximal direction, such that the inner cutter cuts the suture.

Securing the suture with the suture lock at the tissue may include orienting the suture lock perpendicular to the tissue. Securing the suture with the suture lock at the tissue may include orienting the suture lock parallel to the tissue.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows a schematic plan view of a suture anchor assembly for tissue fixation after cinching the suture tight.

FIG. 2A shows a schematic illustration of a suture cinch lock device with a threader assembly according to one embodiment of the present disclosure.

FIG. 2B shows a schematic of a suture being threaded through the open loop of the suture threader line according to one embodiment of the present disclosure.

FIG. 2C shows a schematic of the threader tab being removed from the outer shaft according to one embodiment of the present disclosure.

FIG. 2D shows a schematic of the suture being pulled with the threader tab through the suture cinch lock according to one embodiment of the present disclosure.

FIG. 2E shows a schematic of the suture being pulled with the threader tab through the suture cutter according to one embodiment of the present disclosure.

FIG. 2F shows a schematic of a suture locking cinch device being approximated to the proximal side of the tissue at the location of the defect being repaired according to one embodiment of the present disclosure.

FIG. 2G shows a schematic of a suture locking cinch device having the Lock Pull tab being removed according to one embodiment of the present disclosure.

FIG. 2H shows a schematic of a suture locking cinch device applying force to pull the wire by squeezing the handle of the suture locking cinch device according to one embodiment of the present disclosure.

FIG. 2I shows a schematic of a suture locking cinch device being removed according to one embodiment of the present disclosure.

FIG. 2J shows a schematic of a suture lock in a perpendicular orientation relative to the surface of the tissue according to one embodiment of the present disclosure.

FIG. 2K shows a schematic of a suture lock in a parallel orientation relative to the surface of the tissue according to one embodiment of the present disclosure.

FIG. 3 shows steps in an embodiment of a method for surgically cinching and terminating a monofilament suture anywhere in the body according to one embodiment of the present disclosure.

FIG. 4 shows a drawing and parts description of an embodiment of a suture cinch locking device according to one embodiment of the present disclosure.

FIG. 5A shows drawings and parts description of an embodiment of a suture lock tip assembly according to one embodiment of the present disclosure.

FIG. 5B shows additional drawings and parts description of an embodiment of a suture lock tip assembly according to one embodiment of the present disclosure.

FIG. 5C shows additional drawings and parts description of an embodiment of a suture lock tip assembly according to one embodiment of the present disclosure.

FIG. 5D shows additional drawings and parts description of an embodiment of a suture lock tip assembly according to one embodiment of the present disclosure.

FIG. 5E shows additional drawings and parts description of an embodiment of a suture lock tip assembly according to one embodiment of the present disclosure.

FIG. 5F shows additional drawings and parts description of an embodiment of a suture lock tip assembly according to one embodiment of the present disclosure.

FIG. 5G shows additional drawings and parts description of an embodiment of a suture lock tip assembly according to one embodiment of the present disclosure.

FIG. 6A shows drawings of an embodiment of inner cutter assembly according to one embodiment of the present disclosure.

FIG. 6B shows drawings of an embodiment of outer cutter assembly according to one embodiment of the present disclosure.

FIG. 6C shows drawings of an embodiment of a suture lock assembly according to one embodiment of the present disclosure.

FIG. 6D shows drawings of an embodiment of a suture lock tip assembly according to one embodiment of the present disclosure.

FIG. 7A shows a drawing of an alternate embodiment of lock retention of a flat top with friction lock retention before engaging the lock retention according to one embodiment of the present disclosure.

FIG. 7B shows a drawing of an alternate embodiment of lock retention of a flat top with friction lock retention after engaging the lock retention according to one embodiment of the present disclosure.

FIG. 7C shows a drawing of an alternate embodiment of lock retention of a dome top with reverse lock retention before engaging the lock retention according to one embodiment of the present disclosure.

FIG. 7D shows a drawing of an alternate embodiment of lock retention of a dome top with reverse lock retention after engaging the lock retention according to one embodiment of the present disclosure.

FIG. 8A shows a drawing of a view of an embodiment of a threader assembly according to one embodiment of the present disclosure.

FIG. 8B shows a drawing of another view of an embodiment of a threader assembly according to one embodiment of the present disclosure.

FIG. 8C shows a drawing of a close up view of an embodiment of a threader assembly according to one embodiment of the present disclosure.

FIG. 8D shows another drawing of a close up view of an embodiment of a threader assembly according to one embodiment of the present disclosure.

FIG. 9A shows drawings of an embodiment of an outer lock with alignment according to one embodiment of the present disclosure.

FIG. 9B shows drawings of an embodiment of an outer cutter with alignment according to one embodiment of the present disclosure.

FIG. 9C shows drawings of an embodiment of an assembly with alignment according to one embodiment of the present disclosure.

FIG. 10A shows a drawing of a view of an embodiment of an alternate inner cutter with tabs according to one embodiment of the present disclosure.

FIG. 10B shows a drawing of another view of an embodiment of an alternate inner cutter with tabs according to one embodiment of the present disclosure.

FIG. 11 shows drawings of parts of an embodiment of threaded pull rod tension adjustment according to one embodiment of the present disclosure.

FIG. 12 shows steps in an embodiment of a method of operation of a suture locking cinch system according to one embodiment of the present disclosure.

### DESCRIPTION OF ILLUSTRATIVE EMBODIMENTS

The present suture locking cinch system can be used for surgically cinching and terminating a suture anywhere in the body. This suture locking cinch system includes a instrument that places a suture lock to secure the suture. The suture lock is necessary when a suture is passed through two or more pieces of tissue, and requires a locking device in place of tying knots. This can be very useful in tight spaces where knot tying can be very difficult and troublesome. Specifically, for endoscopic surgery, a suture (e.g., a monofilament type suture) emanating from inside the body is threaded through a suture lock at a distal tip of the instrument, and the instrument is placed down an endoscope. The suture lock is forwarded to the locking site and with the suture being pulled taut, a handle assembly is depressed which deploys the suture lock (e.g., locking mechanism). The locking mechanism is severed from the instrument shaft and the suture is subsequently cut all in one motion. The suture lock includes two pieces of Peek plastic that are assembled to form an interference fit.

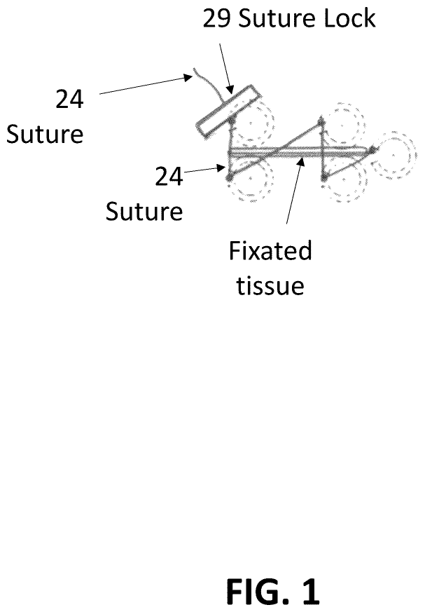

FIG. 1 shows a schematic plan view of an anchor assembly for tissue fixation after cinching the suture tight. A suture 24 (e.g., flexible strand) connecting the anchor assembly is shown on a proximal side (above the plane of the page) of the tissue, and the anchor assembly is positioned on the distal side (below the plane of the page) of a tissue. The anchor assembly has been drawn together to close and fixate the opening by pulling the suture 24 taut.

While not shown in FIG. 1, the suture 24 actually extends up a surgical endoscope for several feet from the tissue up through the endoscope and out of the endoscope into a surgical area. The suture 24 is pulled by a member of a surgical team to make it taut.

By pulling the suture 24 taut, the exposed edges of the opening in the tissue are drawn together and the opening is fixated (e.g., joined). The opening has been closed by pulling the suture 24 tight. A suture lock 29 is engaged with the suture 24 to lock the suture 24 and prevent the fixated tissue from pulling apart. The suture lock 29 is a locking mechanism to lock a suture 24 in place and prevent the suture 24 from being loosened by the tension caused by the anchor assembly having been drawn together. The suture lock 29 may be used to lock any type of suture, whether the suture 24 connects an anchor assembly, or whether the suture 24 is used in a single closure.

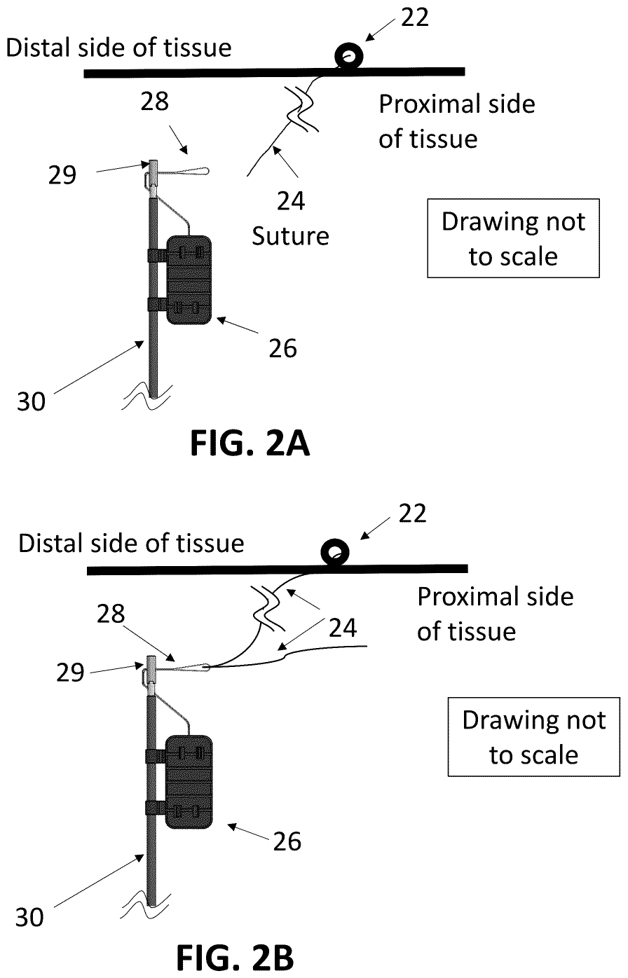

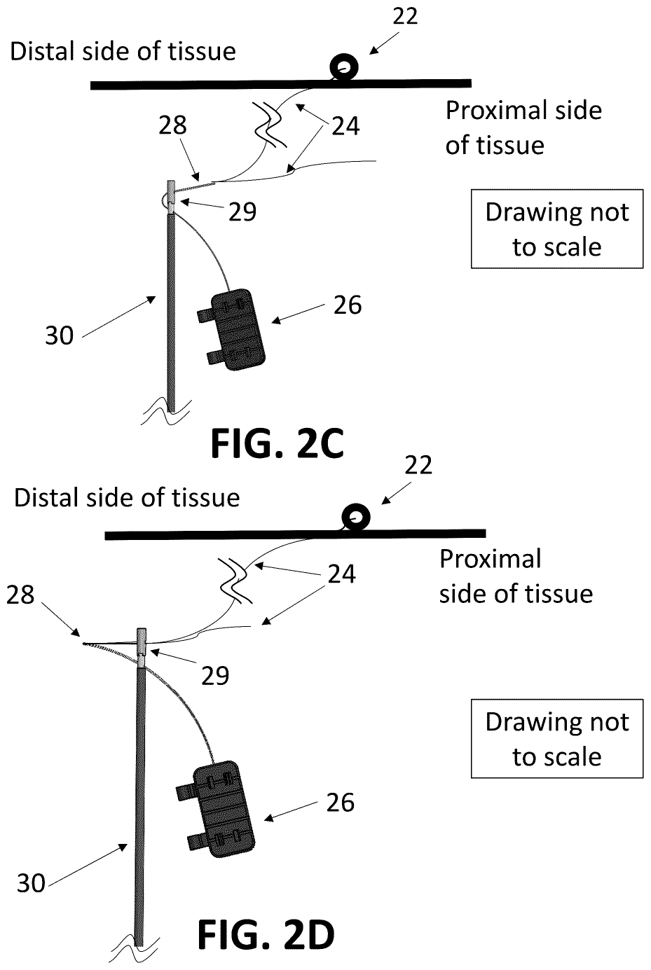

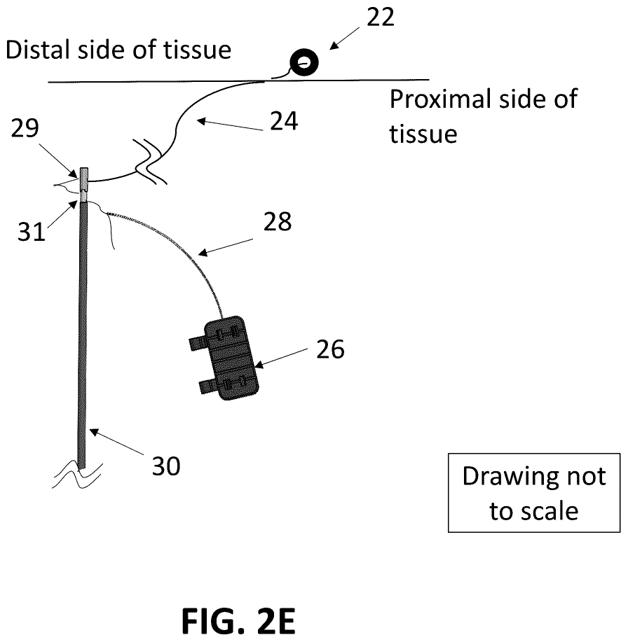

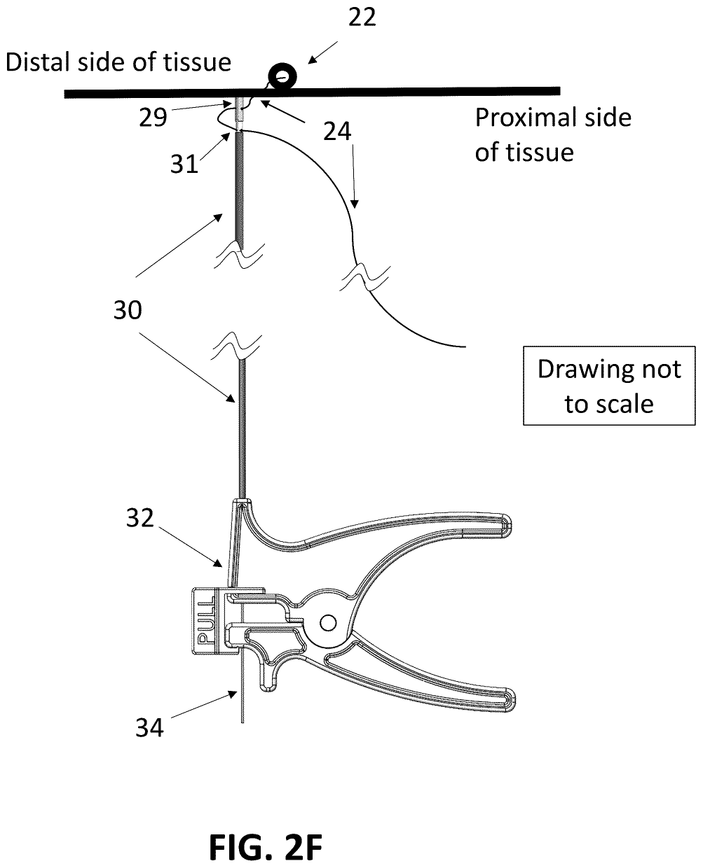

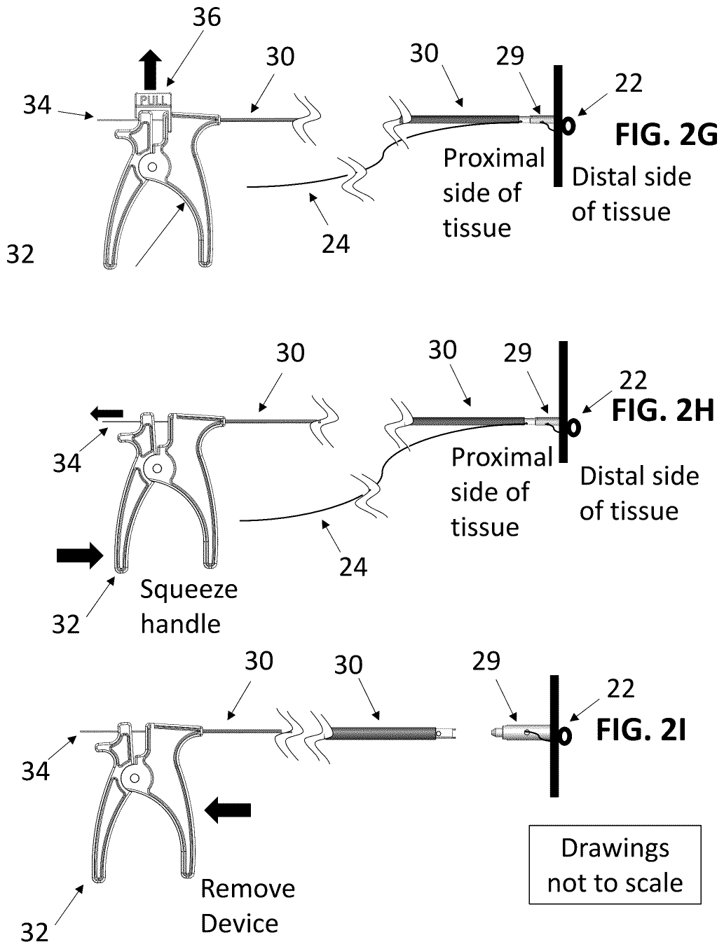

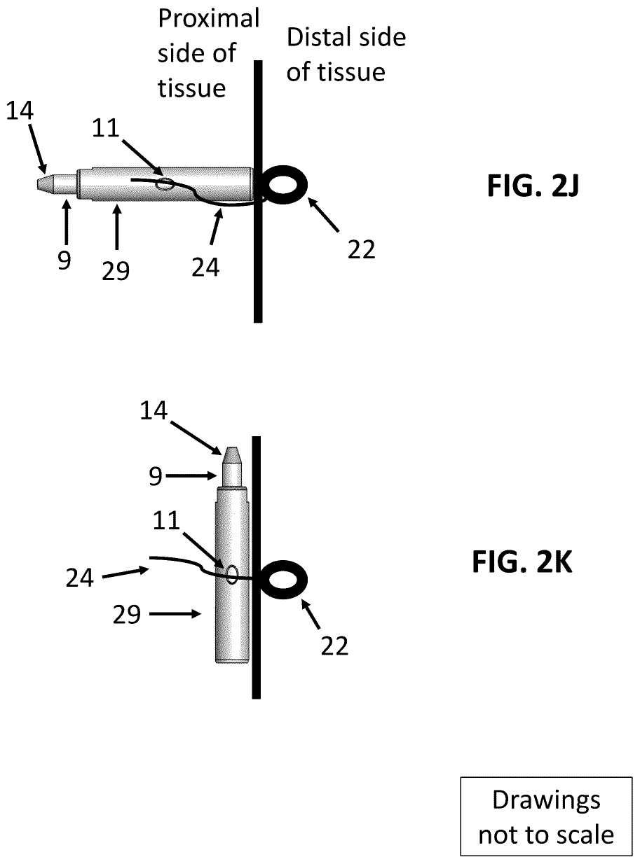

FIGS. 2A through 2K illustrate an embodiment of how the suture cinch lock device 20 may be used to secure a suture lock 29 on a suture 24, including approximating it to the tissue, cinching it, and locking the suture lock 29.

FIG. 2A shows a schematic drawing of a side view of a suture cinch lock device 20 with a threader tab 26. A length of the suture 24 remains after the final anchor 22 has been positioned on the distal side of the tissue. As described above, the suture 24 extends several feet in length from the final anchor 22 through the endoscope out to the surgical team. Thus, the threader tab 26 with a suture threader line 28 may be brought to the suture 24 outside of the proximal end of the endoscope. The threader tab 26 is removably mounted to an outer shaft 30, and the suture threader line 28 is threaded through the suture lock 29.

FIG. 2B shows a schematic drawing of the suture 24 being threaded through an open loop of the suture threader line 28. The threading of the suture 24 through the suture threader line 28 is performed by a member of the surgical team.

FIG. 2C shows a schematic drawing of the threader tab 26 being removed from the outer shaft 30. The threader tab 26 is snapped off of the outer shaft 30.

FIG. 2D shows a schematic drawing of the suture 24 being pulled with the threader tab 26 (e.g., suture tab device) through the suture lock 29. By threading the suture 24 through the suture threader line 28, the suture 24 may be pulled through openings in the suture lock 29. The openings in the suture lock 29 will be described in detail below.

FIG. 2E shows a schematic drawing of the suture 24 being pulled with the threader tab 26 through the suture cutter 31. Note that the drawing is not to scale, and breaks in the continuous suture 24 and outer shaft 30 indicate that the suture 24 and outer shaft have been truncated. The pulling of the suture 24 is being performed in the surgical area by a member of the surgical team.

FIG. 2F shows a schematic drawing of a suture lock 29 being approximated to the proximal side of the tissue at the location of the defect being repaired. The suture lock 29 is at the distal end of the outer shaft 30 and a handle assembly 32 is at the proximal end of the outer shaft 30. At this point in the procedure to place the suture lock 29 at the tissue, the outer shaft 30, having the suture lock 29 at its distal end, is fed into the endoscope, and is pushed down the endoscope until it reaches the tissue to be repaired. Depending on the exact location of the tissue repair, the length of the outer shaft 30 may be between 24 inches and 120 inches. While the suture lock 29 is being pushed into the endoscope, the suture 24 is being pulled taut to enable the suture lock 29 to slide down the suture 24 with a minimum of resistance. The final anchor 22 on the distal side of the tissue provides physical resistance to the tension on the suture 24 while the suture lock 24 is pushed down the suture 24.

FIG. 2G shows a schematic of a lock pull 36 being removed from the handle assembly 32 (e.g., suture cinch). Tension is being applied to the suture 24 by surgical staff. The suture lock 29 is approximated at the proximal side of the tissue at the location of the defect being repaired. As described above, the handle assembly 32 and the proximal end of the suture 24 are located outside the proximal end of the endoscope in the surgical area and are operated by the surgical staff.

FIG. 2H shows a schematic of a handle assembly 32 applying force to the pull wire (e.g., pull rod) 34. Squeezing the handles of the handle assembly 32 pulls the tube inner cutter to create a shear force that separates the tube inner cutter from the inner post. In this disclosure, the pull wire is not limited to a solid wire. In some embodiments, the pull wire is a solid rod, or a hollow tube, or the like.

FIG. 2I shows a schematic of a handle assembly 32 removing the outer shaft 30 while the suture lock 29 is fixated at the proximal side of the tissue at the location of the defect being repaired. This suture lock 29 secures the suture 24.

FIG. 2J shows a schematic of a suture lock in a perpendicular orientation relative to the surface of the tissue. The suture 24 connected to the final anchor 22 on the distal side of the tissue is held in the suture lock 29, and the suture lock fixates the final anchor 22.

FIG. 2K shows a schematic of a suture lock in a parallel orientation relative to the surface of the tissue. The suture 24 connected to the final anchor 22 on the distal side of the tissue is held in the suture lock 29, and the suture lock fixates the final anchor 22.

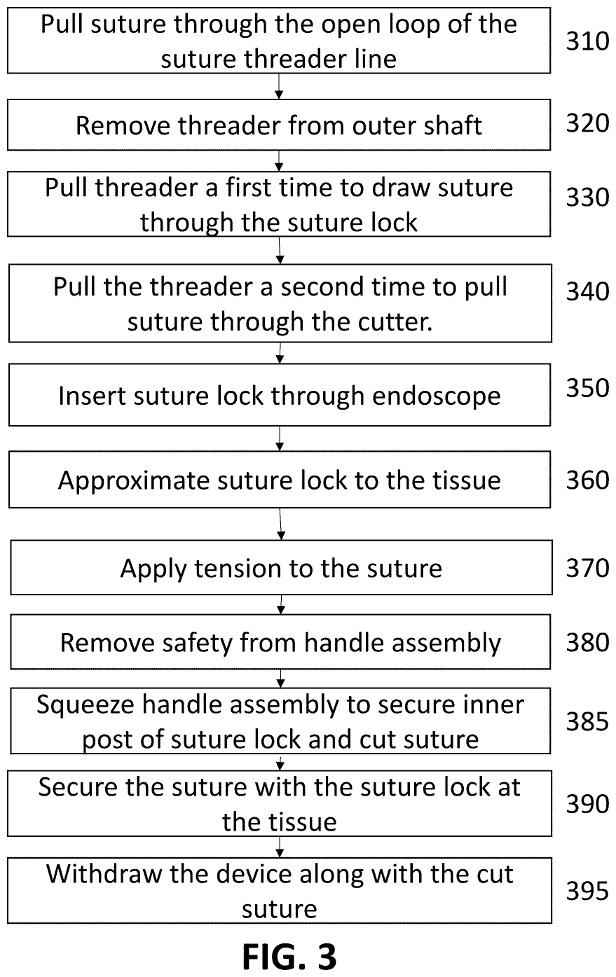

FIG. 3 shows steps in an embodiment of a method for surgically cinching and terminating a monofilament suture anywhere in the body. Prior to initiation of the method, the suture 24 is strung through the endoscope from the final anchor 22 at the distal end of the endoscope to the proximal end of the endoscope outside of the patient. The suture 24 may extend beyond the proximal end of the endoscope by more than 24 inches.

At 310, the suture 24 is pulled through the open loop of the suture threader line 28. As illustrated in FIG. 2B, the suture 24 is threaded through the loop suture threader line 28 outside of the patient in the surgical area by the surgical team. The suture 24 may be a flexible strand. The suture 24 may be a braided polyester suture, or a monofilament strand. In some embodiments, the suture threader line 28 may comprise a monofilament strand, a flexible strand, or a braided polyester suture. In some embodiments, the suture threader line 28 may comprise a metal, such as a steel wire of nitinol.

At 320, the threader tab 26 is removed from the outer shaft 30. As illustrated in FIG. 2C, in some embodiments the threader tab 26 is removed from the outer shaft 30 by unclipping the threader tab 26 from the outer shaft 30. The clips and the threader tab 26 may be fabricated as one part, or may be assembled from more than one part. The clips and the threader tab 26 may be fabricated from a plastic material, a polymeric material, a metal material, or the like. In some embodiments, the threader tab are coupled to the outer shaft 30 with alternate mechanisms.

At 330, the threader tab 26 is pulled a first time to draw the suture 24 through the suture lock 29. As illustrated in FIG. 2D, the suture 24 is held in the loop of the suture threader line 28, and the threader tab 26 is used to pull the suture 24 through the suture lock 29.

At 340, the threader tab 26 is pulled a second time to pull the suture 24 through the suture cutter 31. As illustrated in FIG. 2E, the suture lock tip assembly has the suture cutter 31 built into a suture lock tip assembly 40 that can cut the suture 24 at the appropriate time. The suture 24 is strung through the suture lock 29 and the suture cutter 31, and is pulled taut by the surgical team.

At 350, the suture lock 29 is inserted through the endoscope. As illustrated in FIG. 2F, the suture lock 29 is mounted on the distal end of the outer shaft 30, and the suture lock 29 is placed into the endoscope and pushed down the endoscope to meet the tissue. At the same time the suture lock 29 is being pushed down the endoscope by surgical staff, the surgical staff is pulling the suture 24 to provide tension to counter the downward force being placed on the suture lock 29 as it slides down the suture 24.

At 360, the suture lock 29 is approximated to the tissue. As also illustrated in FIG. 2F, the suture lock 29 is placed against the proximal side of the tissue in close proximity to the final anchor 22, which is on the distal side of the tissue.

At 370, tension is applied to the suture 24.

At 380, the safety is removed. As illustrated in FIG. 2G, the safety (e.g., safety pull tab) 36 is removed from the handle assembly 32. By removing the safety 36, it becomes possible to squeeze the handles of the handle assembly 32 together.

At 385, the handle is squeezed to secure the inner post 9 of the suture lock 29 and cut the suture 24. As illustrated in FIG. 2H, the handle is squeezed and the pull wire 34 applies shear force to tube inner cutter 8 which pulls the inner post 9. The proximal end of the inner post 9 is sheared off of the inner post 9, as illustrated in non-limiting FIG. 2H and FIG. 2I. As the end of the inner post 9 is sheared off of the inner post 9, the suture is severed by the tube outer cutter 7.

At 390, the suture is secured with the suture lock at the tissue.

At 395, the device is withdrawn along with the cut suture 24. As illustrated in FIG. 2I, the handle assembly 32 is withdrawn from the endoscope which removes the outer shaft 30 while the suture lock 29 is fixated at the proximal side of the tissue at the location of the defect being repaired. The suture lock 29 is left at the site of the tissue repair on the proximal side of the tissue.

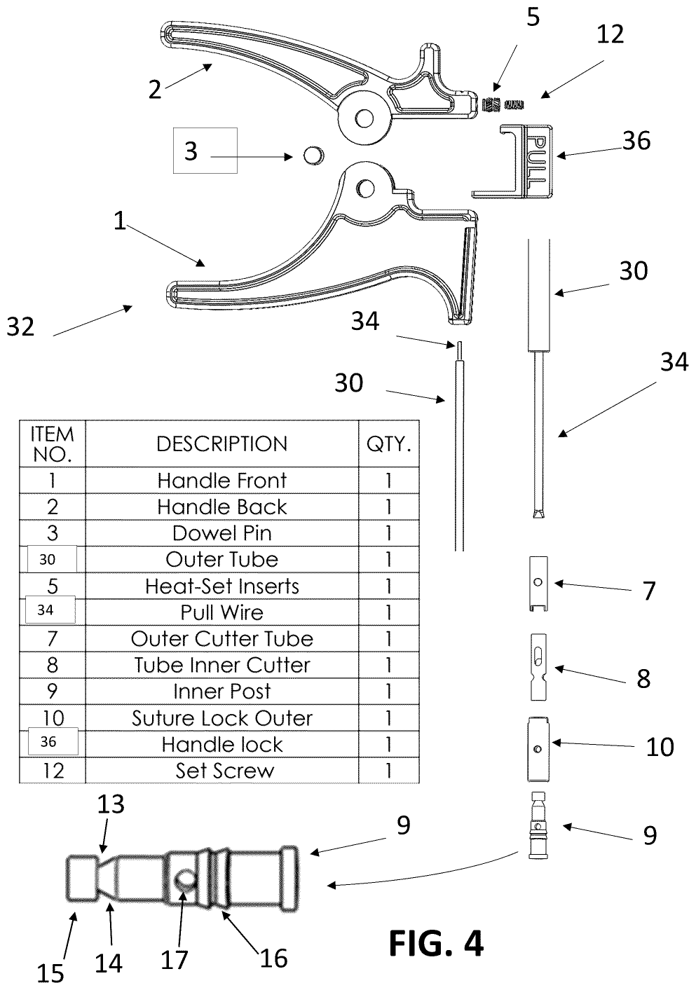

FIG. 4 shows a drawing and parts description of an embodiment of a suture cinch locking device. The suture cinch locking device includes a handle assembly 32. The handle assembly 32 comprises a handle front (e.g., front handle) 1 and a handle back (e.g., back handle) 2. In some embodiments, a dowel pin 3 may be inserted in circular rings in the handle front 1 and handle back 2 to hold the two handles together and to provide a pivot point for the handles. In some embodiments, the handles could also be molded with a boss feature that snaps together in place of the dowel pin for a pivot, configured to movably join the handle front to the handle back and to provide a pivot hinge about which the handle front and handle back rotate. In some embodiments, a dowel pin may be configured to movably join the handle front to the handle back.

Heat set inserts 5 may be pressed into the top of the handle back 2. A set screw 12 is threaded into the heat set inserts. A safety (e.g., handle lock) 36 can be positioned between the handle front 1 and the handle back 2. The safety 36 prevents accidental closing (e.g., squeezing) of the handles of the handle assembly 32.

The suture locking cinch device also includes an outer shaft 30 and a pull wire 34. The pull wire 34 is positioned within the outer shaft 30 and feeds through a channel in the handle front 1 through to the heat set insert 5 on the handle back 2. The pull wire 34 feeds through the heat set insert 5 and is held in place by friction with the set screw 12. The suture locking cinch device also includes an outer cutter tube 7 that fits over a tube inner cutter 8, and an inner post 9 that fits in a suture lock outer 10.

The inner post 9 is designed to secure the suture 24 and to allow the suture lock 29 to breakaway from the rest of the suture locking cinch device. The inner post 9 has a shear point 13 that is where a breakaway portion 15 meets a shearing undercut 14. Though not shown here, in operation, tabs on an inside surface of the tube inner cutter 8 interlock with the breakaway portion 15 at the shear point 13. Ribs 16 on the inner post 9 provide resistance to the tension applied in a proximal direction on the breakaway portion 15. The suture threader line 28 is pulled through an inner post feedthrough 17 when the suture 24 is pulled through the suture lock 29, as illustrated in FIG. 2D.

FIGS. 5A through 5G shows drawings and parts description of an embodiment of a suture lock tip assembly 40. FIG. 5A shows components comprising the suture lock tip assembly 40, including the outer shaft 30, and the pull wire 34 slidably positioned inside of the outer shaft 30. A tube inner cutter 8 is slidably inserted into an outer cutter tube 7. The inner post 9 is slidably inserted into the suture lock outer 8.

The opening in outer cutter tube 7 is an outer cutter feedthrough 18. The opening in the tube inner cutter 8 is an inner tube cutter feedthrough 19. The opening in the suture lock outer 10 is a suture lock outer feedthrough 11. The opening in the inner post 9 is an inner post feedthrough 17.

FIG. 5B shows the assembled suture lock tip assembly 40 as a non-transparent solid. The suture cutter 31 is shown fixed in the outer shell 30. The suture lock 29 with the suture lock outer 10 is apparent. The opening visible in the outer cutter tube 7 is an outer cutter feedthrough 18. The suture threader line 28 is pulled through the outer cutter feedthrough 18 when the suture 24 is pulled through the suture cutter 31, as illustrated in FIG. 2E.

FIG. 5C shows another view the assembled suture lock tip assembly 40 as a partially transparent solid. The outer shell 30 and the suture lock 29 are shown as being partially transparent to show the pull wire 34 under the outer shell 30, and to show the inner post 9 and a portion of the inner tube cutter 8 positioned in the suture lock outer 10.

FIG. 5D shows another view the assembled suture lock tip assembly 40 as a partially transparent solid with the outer cutter tube 7 as partially transparent along with the partially transparent outer shell 30 and the suture lock outer 10. The inner tube cutter 8 becomes visible with the outer cutter tube 7 made partially transparent. The opening in the inner tube cutter 8 is the inner tube cutter feedthrough 19. The outer cutter feedthrough 18 lines up concentrically with the inner tube cutter feedthrough 19 to allow the suture 24 to be pulled through the suture cutter 31, as illustrated in FIG. 2E.

FIG. 5E shows another view of the assembled suture lock tip assembly 40 as a partially transparent solid. In addition to the parts of the suture lock tip assembly 40 that have already been made partially transparent, FIG. 5E has also made the tube inner cutter 8 partially transparent. The inner post 9 can be seen, and it includes the shear point 13, where the breakaway portion 15 meets the shearing undercut 14, as well as the ribs 16 and the inner post feedthrough 17. The wire pull 34 is being pulled by the handle assembly 32 to place shear tension on the shear point 13 on the inner post 9.

FIG. 5F shows another view of the assembled suture lock tip assembly 40 as a partially transparent solid. A pulling force 6 is being applied to the pull wire 34 by the handle assembly 32. The breakaway portion 15 on the inner post 9 is being pulled away from the shearing undercut 14. Tabs on an inner surface of the tube inner cutter 8 interlocked with the breakaway portion 15 and placed a shearing force on the shear point 13 causing the breakaway portion 15 to be sheared off of the inner post 9. Further, the inner post 9 has been moved proximally inside of the suture tip assembly to put the shearing undercut outside of the suture lock outer 10.

FIG. 5G shows another embodiment of the assembled suture lock tip assembly 40 as a partially transparent solid. In this embodiment, the inner post 9 has a dome on the distal of the inner post 9. The dome on the distal end of the inner post 9 may increase the ease of inserting the suture lock 29 and outer shaft 30 into the endoscope and pushing the suture lock 29 and outer shaft 30 down the endoscope to the tissue.

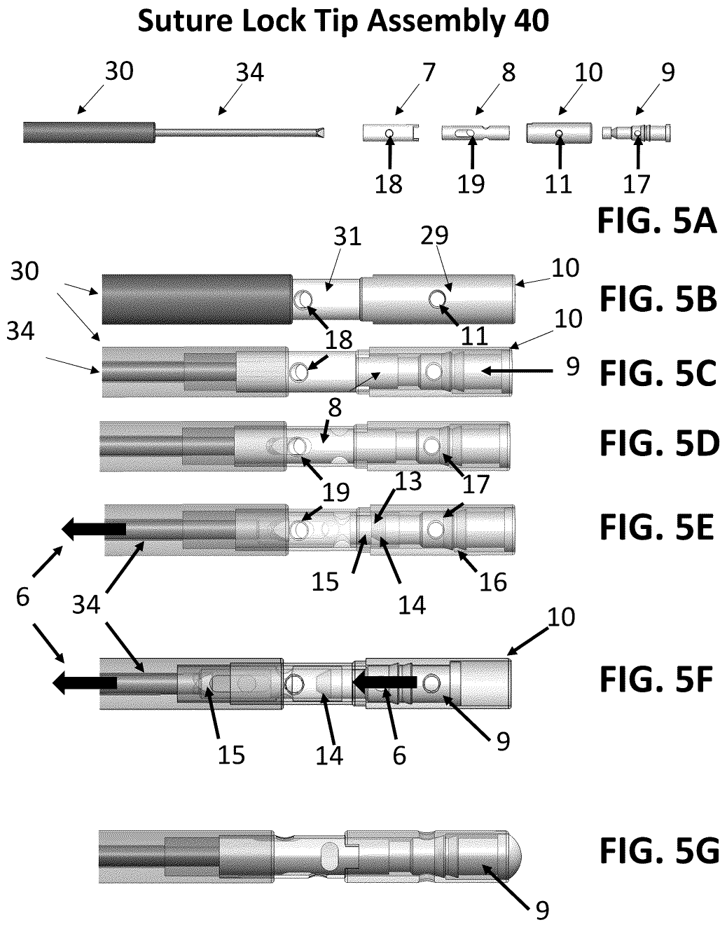

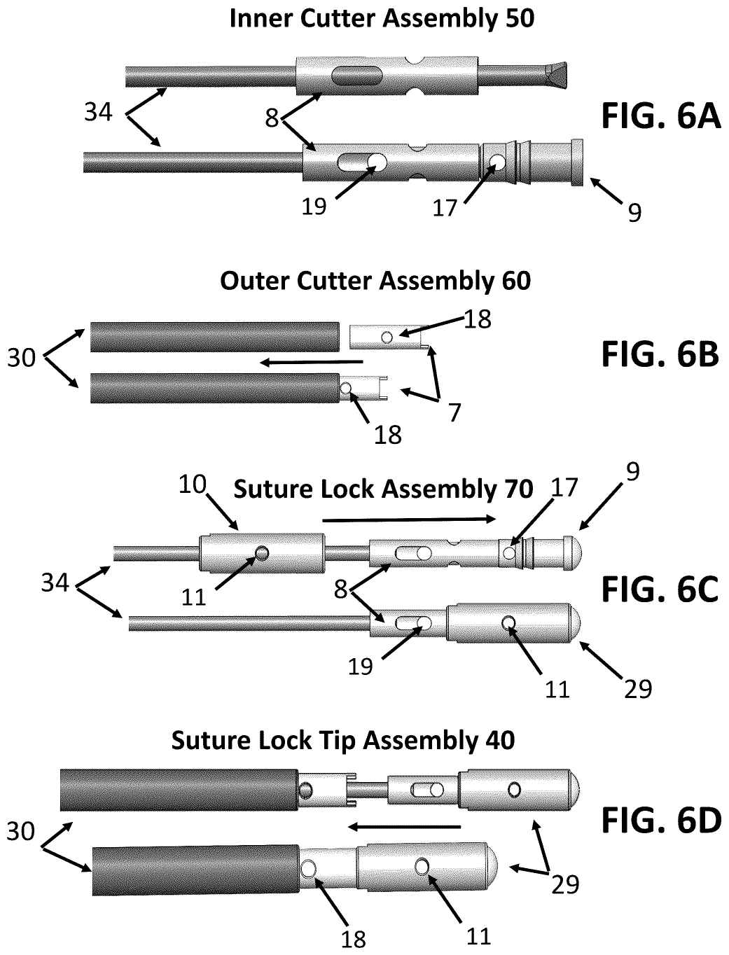

FIG. 6A shows drawings of an embodiment of an inner cutter assembly 50 before and after the inner post 9 is inserted into the tube inner cutter 8. The inner post 9 is slidably inserted along the wire pull 34 into the tube inner cutter 8. The inner post feedthrough 17 and the inner tube cutter feedthrough 19 are shown.

FIG. 6B shows drawings of an embodiment of an outer cutter assembly 60 before and after the outer cutter tube 7 is inserted into the outer shaft 30. The outer cutter feedthrough 18 is shown.

FIG. 6C shows drawings of an embodiment of a suture lock assembly 70 before and after the suture lock outer 10 is pushed distally onto the tube inner cutter 8 and the inner post 9. In this embodiment, the inner post 9 has a dome on the distal end. When the tube inner cutter 8 and the inner post 9 are covered by the suture lock outer 10, the suture cutter 31 is complete. When the suture lock outer 10 is positioned over the inner post 9, the suture lock outer feedthrough 11 aligns with inner post feedthrough 17 to provide an opening through the suture lock 29.

FIG. 6D shows drawings of an embodiment of a suture lock tip assembly 40 before and after the suture lock assembly 70 is pushed into the outer cutter assembly 60. After the assembled suture lock assembly 70 is pushed into the outer cutter assembly 60, the inner post feedthrough 17 is aligned with the suture lock outer feedthrough 11 to form an opening through the suture lock 29. After the assembled suture lock assembly 70 is pushed into the outer cutter assembly 60, the inner tube cutter feedthrough 19 is aligned with the outer cutter tube feedthrough 18. In the assembled suture lock assembly 70, the suture lock outer feedthrough 11 and the outer cutter tube feedthrough 18 align along a lengthwise axis of the suture lock assembly 70.

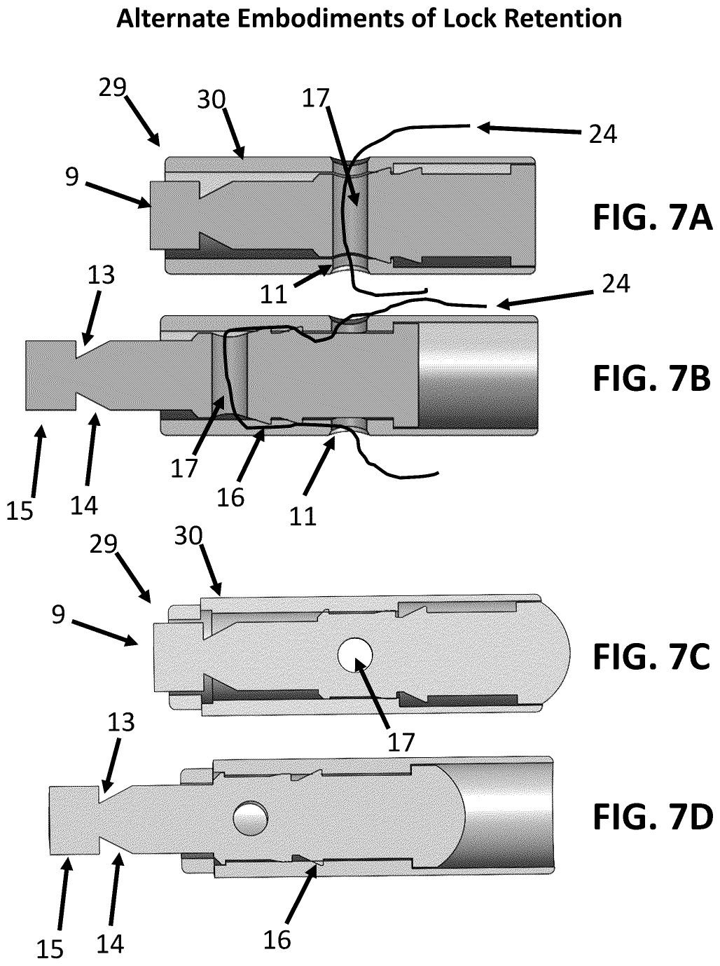

FIG. 7A shows a drawing of an alternate embodiment of lock retention of a flat top with friction lock retention before engaging the lock retention. The inner post 9 is seated in the suture lock outer 10 such that the inner post feedthrough 17 is aligned with the suture lock outer feedthrough 11 to form an opening through the suture lock 29. A suture 24 is threaded through the aligned inner post feedthrough 17 and suture lock outer feedthrough 11. This schematically illustrates the condition of FIG. 2D where the suture 24 is pulled through the suture lock 29.

FIG. 7B shows a drawing of an alternate embodiment of lock retention of a flat top with friction lock retention after engaging the lock retention. The lock retention is engaged when the inner post 9 is pulled by the wire pull 34, as schematically illustrated in FIG. 5F. Pulling the inner post 9 proximally causes the ribs 16 to engage with an inner surface of the outer shell 30 and locks (e.g., retains) the suture 24 between the ribs 16 and the inner surface of the outer shell 30.

FIG. 7C shows a drawing of an alternate embodiment of lock retention of a dome top with reverse lock retention before engaging the lock retention. The suture lock 29 of FIG. 7C is rotated 90 degrees relative to the suture lock 29 of FIG. 7A. The inner post 9 is seated in the suture lock outer 10 such that the inner post feedthrough 17 is aligned with the suture lock outer feedthrough 11 to form an opening through the suture lock 29.

FIG. 7D shows a drawing of an alternate embodiment of lock retention of a dome top with reverse lock retention after engaging the lock retention. The suture lock 29 of FIG. 7D is rotated 90 degrees relative to the suture lock 29 of FIG. 7B. The inner post 9 is seated in the suture lock outer 10 such that the inner post feedthrough 17 is aligned with the suture lock outer feedthrough 11 to form an opening through the suture lock 29. The lock retention is engaged when the inner post 9 is pulled by the wire pull 34, as schematically illustrated in FIG. 5F. Pulling the inner post 9 proximally causes the ribs 16 to engage with the inner surface of the outer shell 30 and locks (e.g., retains) the inner post 9 with the ribs 16 against the inner surface of the outer shell 30.

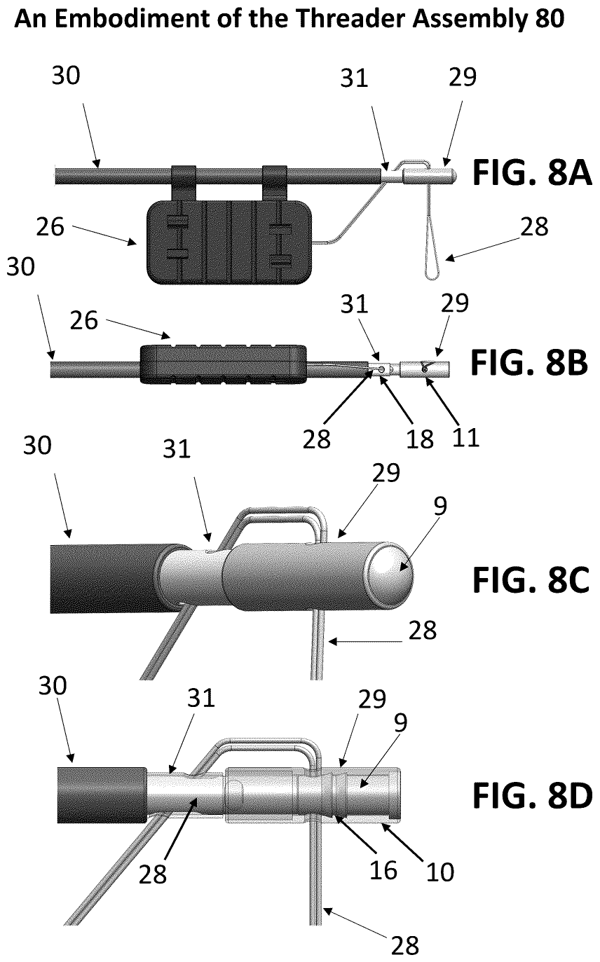

FIG. 8A shows a drawing of another view of an embodiment of a threader assembly 80. The threader tab 26 is coupled to the outer shaft 30 and has the suture threader line 28 threaded through the suture cutter 31 and the suture lock 29.

FIG. 8B shows a drawing of another view of an embodiment of a threader assembly 80. The threader assembly 80 shown in FIG. 8B is rotated 90 degrees from that of FIG. 8A. The suture threader line 28 is threaded up through the outer cutter feedthrough 18 in the suture cutter 31 and back through the suture lock outer feedthrough 11 in the suture lock 29.

FIG. 8C shows a drawing of a close up view of an embodiment of a threader assembly 80. The close up shows the suture threader line 28 is threaded up through the outer cutter feedthrough 18 in the suture cutter 31 and back through the suture lock outer feedthrough 11 in the suture lock 29. In this embodiment, the inner post 9 has a dome on the distal end of the inner post 9.

FIG. 8D shows another drawing of a close up view of an embodiment of a threader assembly 80. The close up shows the suture lock outer 10 and the outer cutter tube 7 as partially transparent solids. The suture lock 29 and the suture cutter 31 are shown as being partially transparent to show the inner post 9, the ribs 16, and the tube inner cutter 8. The suture threader line 28 is shown threading up through the suture cutter 31 and back down through the suture lock 29.

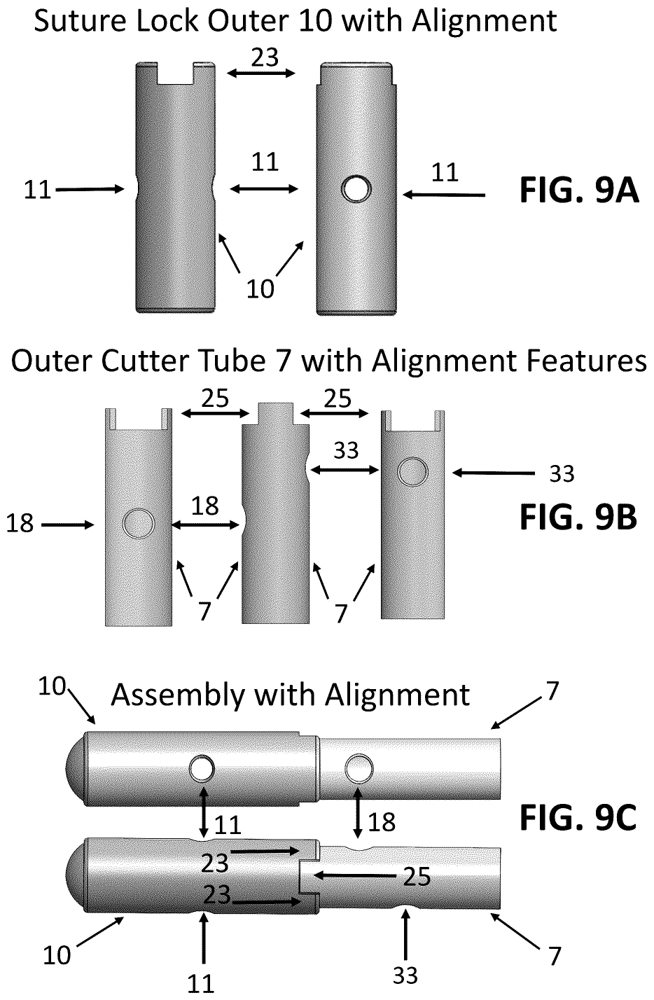

FIG. 9A shows drawings of an embodiment of a suture lock outer 10 with alignment features 23. Two orientations of the suture lock outer 10 are shown rotated 90 degrees from each other. The suture lock outer feedthrough 11 is shown in both orientations. Suture lock outer alignment features 23 operate in the assembly of the suture lock 29 to secure the suture lock outer 10 to the outer cutter tube 7.

FIG. 9B shows drawings of an embodiment of an outer cutter tube 7 with alignment features 25. Outer cutter tube alignment features 25 cooperate with the suture lock outer alignment features 23 to secure the suture lock outer 10 to the outer cutter tube 7. A first opening in the outer cutter tube 8 is an outer cutter tube feedthrough 18, and a second opening is a second inner tube cutter feedthrough 33.

FIG. 9C shows drawings of two orientations of an embodiment of an assembly with alignment features engaged. The suture lock outer 10 and the outer cutter tube 7 are coupled with the suture lock outer alignment features 23 aligned with the outer cutter tube alignment features 25. The two orientations of the assembly are shown rotated 90 degrees from each other. The suture lock outer feedthrough 11 is shown in both orientations. The alignment features operate in the assembly of the suture lock outer 10 to the outer cutter tube 7 to align the suture lock outer feedthrough 11 and the outer cutter feedthrough 18.

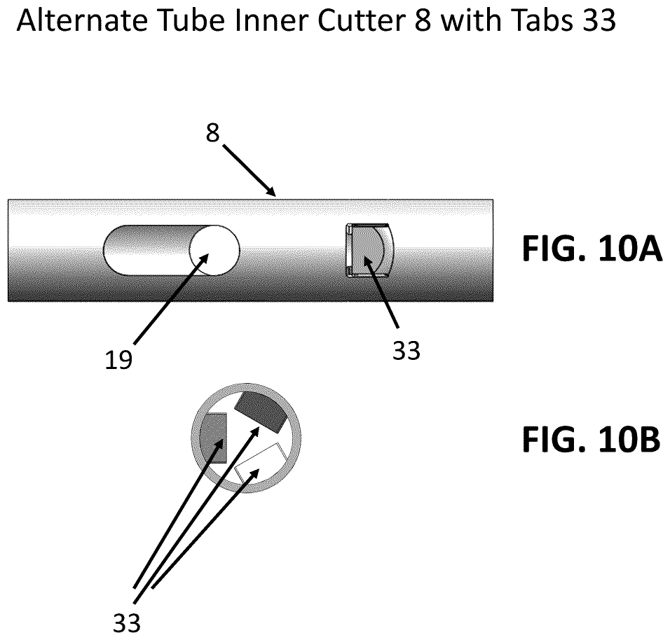

FIG. 10A shows a drawing of a view of an embodiment of an alternate inner cutter with tabs 33. The tabs 33 are on the inner surface of the tube inner cutter 8. Additionally, the inner tube cutter 8 has an inner tube cutter feedthrough 19.

FIG. 10B shows a drawing of another view of an embodiment of an alternate inner cutter with tabs. FIG. 10B shows a cross section view of the of the embodiment of the inner tube cutter 8 in FIG. 10A. In some embodiments, there may be two, three, or four tabs 33.

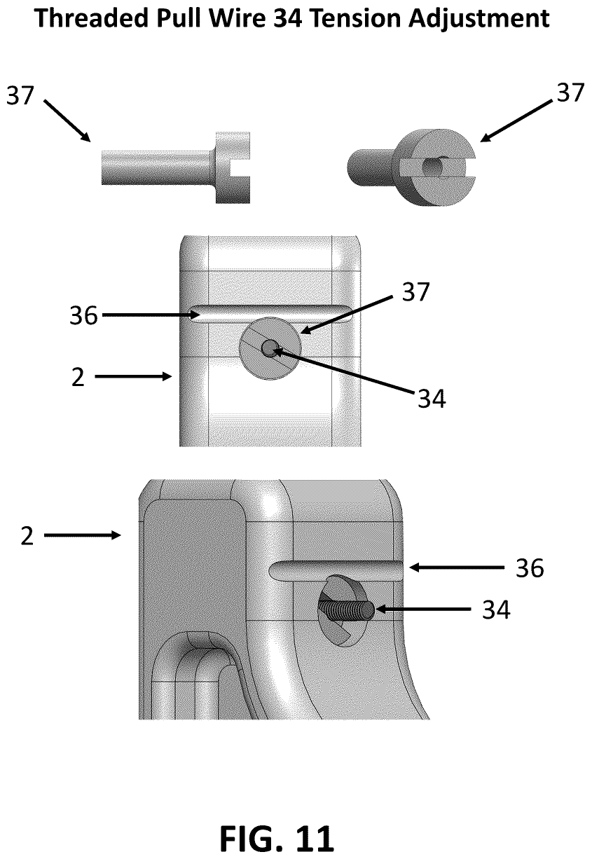

FIG. 11 shows drawings of parts of some of the threaded pull wire tension adjustment according to some embodiments of the present disclosure. FIG. 11 is a blowup of a portion of the handle back 2 that includes the pull wire tension adjustment portion 37. The pull wire 34 feeds through the front handle 1 and extends from the back handle 2. A wire securing portion 37 is threaded onto the threaded mounting fixture 35 and tightened onto the pull wire to provide friction fixation of the pull wire. A groove 36 for seating a ridge on the safety 36 is formed in a portion of the front handle 1.

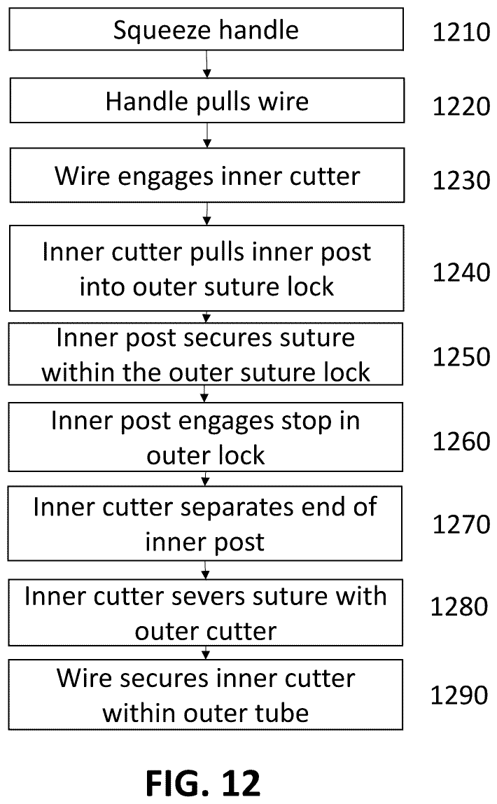

FIG. 12 shows steps in an embodiment of a method of operation of a suture locking cinch device.

At 1210, the handle is squeezed. The handle assembly 32 is squeezed to provide tension to the pull wire 34.

At 1220, the handle pulls the wire. Squeezing the handle assembly 32 pulls the wire 34 that is coupled to the tube inner cutter 8. A non-limiting illustration of the pull wire 34 being pulled is shown in FIG. 5E.

At 1230, the wire engages the inner cutter. The tube inner cutter 8 is engaged by wire 34 and the force created by squeezing the handle assembly 32 pulls the tube inner cutter 8 proximally.

At 1240, the tube inner cutter pulls the inner post 9 into the suture outer lock 10. The pulling of the wire 34 forces the inner post 9 to move proximally into the suture lock outer 10. A pulling force 6 is being applied to the pull wire 34 by the handle assembly 32. Tabs 33 on the inner surface of the tube inner cutter 8 interlock with the breakaway portion 15. The breakaway portion 15 on the inner post 9 is being pulled away from the shearing undercut 14.

At 1250, the inner post secures the suture within the outer suture lock. The proximal movement of the inner post 9 in the outer suture lock 10 engages the ribs 16 into the inner surface of the outer suture lock 10. The movement of the inner post 9 and the engagement of the ribs 16 also locks the suture 24 between the ribs 16 and the inner surface of the suture outer lock 10.

At 1260, the inner post engages the stop in the outer lock. The proximal movement of the inner post 9 is stopped when the inner post 9 engages the stop in the suture outer lock 10.

At 1270, the inner cutter separates the end of the inner post. The tabs 33 in the tube inner cutter 8 engage the breakaway portion 15 on the inner post 9 and pull the breakaway portion 15 away from the shearing undercut 14, thus separating the inner post 9 from the tube inner cutter 8. The breakaway portion 15 of the inner post 9 can be sheared off of the inner post 9 with about 6 pounds of force at the shear point. The grip force necessary to produce the 6 pounds of shear force at the shear point requires about 12 pounds of grip force produced by squeezing the handle assembly 32. The 12 pounds of force required at the grip handle represents a fraction of the average hand squeezing strength of women (about 65 pounds of force) and men (about 100 pounds of force).

At 1280, the inner cutter severs the suture with the outer cutter. At about the same that the inner post 9 is separated from the tube inner cutter 8, the suture 24 is cut by the tube outer cutter 7.

At 1290, the wire secures the inner cutter within the outer shaft. After separating the breakaway portion 15 from the inner post 9, and severing the suture 24, the outer shaft 30 and the cut suture 24 are withdrawn from the endoscope.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A suture locking cinch system, the system comprising:
a handle assembly, the handle assembly comprising:
a handle front; and
a handle back;
an outer shaft, the outer shaft configured to provide a flexible hollow tube, a proximal end of the outer shaft removably connected to the handle front;
a pull rod, the pull rod removably attached to the handle back and configured to be run through a channel in the handle front and through the outer shaft;
a suture lock tip assembly, the suture lock tip assembly configured to be frangibly removable to a distal end of the outer shaft; and
a threader assembly, the threader assembly configured to be removably coupled to the outer shaft.

2. The system of claim 1, wherein the threader assembly comprises:
a suture threader line, the suture threader line having a closed loop on a distal end the suture threader line; and
a threader tab, a proximal end of the suture threader line configured to be removably attached to a distal end of the threader tab.

3. The system of claim 1, wherein the suture lock tip assembly comprises:
an inner cutter assembly;
an outer cutter assembly; and
a suture lock assembly,
wherein:
the outer cutter assembly is fixed into a distal end the outer shaft;
the inner cutter assembly is mounted in the outer cutter assembly; and
the suture lock assembly is mounted in the inner cutter assembly.

4. The system of claim 3, wherein the suture lock assembly comprises:
a suture lock outer; and
an inner post,
wherein:
the inner post is positioned in the suture lock outer; and
the pull rod is coupled to a proximal end of the inner post.

5. The system of claim 4, wherein the inner post comprises:
ribs, the ribs are configured to provide an interference fit against the suture lock outer;
a breakaway portion;
a shearing under cut; and
a shear point,
wherein the inner post is configured to breakaway at the shear point when a shear force is applied by the pull rod.

6. The system of claim 5, wherein the shear point is configured to breakaway when the shear force exceeds about 6 pounds.

7. The system of claim 3, wherein the inner cutter assembly comprises:
an inner cutter;
the inner cutter comprises at least one tab on an inner surface of inner cutter; and
the at least one tab is configured to engage the breakaway portion of the inner post.

8. The system of claim 1, wherein:
the handle front and handle back are molded with a boss feature that snaps together; and
the boss feature is configured to movably join the handle front to the handle back and to provide a pivot hinge about which the handle front and handle back rotate.

9. The system of claim 1, wherein the handle assembly further comprises:
a dowel pin, the dowel pin configured to movably join the handle front to the handle back and to provide a pivot hinge about which the handle front and handle back rotate.

10. An inner post with a shear point for placement and release of a suture lock, the inner post comprising:
a breakaway portion, the breakaway portion configured to be engaged by a hooking portion;
a shearing undercut, the shearing undercut configured to provide an undercut for defining the breakaway portion; and
a shear point, the shear point located at a junction on the inner post between the breakaway portion and the shearing undercut, the shear point configured to allow the breakaway portion to be separated from the shearing undercut.

11. The inner post of claim 10, further comprising:
an opening in the inner post, the opening passing through the inner post at a right angle to a longitudinal direction of the inner post, the opening configured to allow the passage of a suture through the opening from one side of the inner post to the opposite side of the inner post.

12. The inner post of claim 10, further comprising:
a suture lock outer, the suture lock outer configured to receive the inner post, the inner post configured to be slidably inserted into the suture lock outer.

13. The inner post of claim 12, further comprising:
one or more ribs encircling the inner post, the one or ribs encircle the inner post radially at a right angle to the longitudinal direction of the inner post, the ribs are configured to engage an inside surface of to the suture lock outer.

14. A method of surgically cinching and terminating a suture, the method comprising:
pulling the suture through an open loop of a suture threader line, the suture threader line removably attached to a distal end of a threader tab;
pulling the suture threader line through a suture lock;
inserting the suture lock through an endoscope, the suture lock being positioned at the distal end of an outer shaft;
approximating the suture lock to a tissue;
applying tension to the suture, the tension on the suture is applied by pulling the suture;
removing a safety from a handle assembly;
squeezing the handle assembly to secure an inner post of the suture lock;
separating, frangibly, the suture lock while cutting the suture; and
withdrawing the outer shaft along with the cut suture.

15. The method of claim 14, wherein the pulling the suture through an open loop of a suture threader line is performed outside of an endoscope.

16. The method of claim 14, wherein squeezing the handle assembly pulls the inner post in a proximal direction, such that:
a shear force is created at a shear point in the inner post that separates a breakaway portion of the inner post from a shearing under cut of the inner post at the shear point; and
the inner cutter cuts the suture.

17. The method of claim 14, wherein securing the suture with the suture lock at the tissue comprises orienting the suture lock perpendicular to the tissue.

18. The method of claim 14, wherein securing the suture with the suture lock at the tissue comprises orienting the suture lock parallel to the tissue.
